SEQUENCE LISTING (26 sequences)

SEQ ID NO: 1 - DNA, 31 bp, Artificial Sequence
  Description of Artificial Sequence primer
gcgagctcagcgcgagtgactggatatcgga

SEQ ID NO: 2 - DNA, 31 bp, Artificial Sequence
  Description of Artificial Sequence primer
gcggtaccactgtataaatagcaaaggcaac

SEQ ID NO: 3 - DNA, 2964 bp, Methylophilus methylotrophus
  CDS (684)..(2930)
gatatcggaatgagcattaagtctgacaaatggatacgcagaatggctgaacaacacggcatgattgagccgtttgagcccaagcttgtacgtgagaccaatggccagaagattgtttcttatggcacctcttcttacggttacgatatccgttgtgctgacgaattccgcgtatttaccaatatcaacagcaccatagttgaccccaagcaatttgacccgcagtcgtttgtcgaggtctccggcaaaggctattgcgtgattccccctaactcatttgcactggcgcgcacggtagagtatttccgtattcctcgctctgtactgactgtatgcctcggcaagtcgacttatgcgcgttgcggcattatcgtcaacgtcaccccctttgaaccagagtgggaaggctatgtcacactagagttcagcaacaccacaccgctacccgccaaaatttatgctggcgaaggctgtgcgcaagtgctgttctttgagtctgatgaaatctgtgaaacgagctacaaagaccgtggtggtaaataccagggtcaaattggcgtgaccctgccaaaaatataacggcaacattgaacaataacctgacattcaccaagggcacggtgcaaagcaaatgctttctctgtgcccttgtgtcttgattttagcggtaaaggatttattgcatgaaatttagattccctatcgtcattattgacgaggacttccgctccgagaactcttccggcctgggcatccgtgtgctggcgaaagccatcgaagatgagggcctggaagtgcttggcgtcaccagctatggcgacctgacctctttcgcccagcagcaaagccgtgcatcagcctttatcctgtcgattgatgatgaggaaatcgttgaggagaaaccggaagccattgagcaactgcgtaactttgtgcaggaaatccgttaccgcaacgaggaaatccccattttcctgcatggcgaaacccgtaccagccgtcacatccctaacgatgtgttgcgcgagttgcacggctttatccatatgaatgaagacacgcctgagtttgtggcgcgcctgattatccgcgaagccaaagcctacctggacagcttgccaccgcccttcttcaaggcactcactcattacgcggctgatggctcttattcatggcactgtcctggtcactcgggtggcgtagcctttctgaaatccccagtcgggcagatgttccaccagttttttggcgagaacatgctgcgtgcagacgtgtgtaatgcggtagatgaattaggccaattactggatcacaccggcccggtggccgcttctgagcgcaacgctgcgcgcatctacaactgcgaccatttgtactttgtcactaacggcacctcaacatcgaacaagattgtctggaactcaaccgtggcgccgggtgatattgtagtggttgatcgtaactgccataaatccgtattgcactccatcattatgacgggtgccgtgcccgtgttcctgatgccaacgcgcaaccatttcggcattatcgggcctatcccaaaaagtgaattcgcctgggaaaacatccagaaaaagatcgcacgcaacccgtttgccaccgacaaaaatgccaagccacgcgtgctgaccattacacagtccacctatgatggcgtgttgtataacgtggaagaaatcaaggaaatgctggatggcaaaattgacaccctgcactttgacgaagcctggttgccacatgcgaccttccatgacttttatggtgactaccatgcgattggcgctgaccgcccacgctgtaaagaatccatggtgttctccacccagtccacgcacaaactattggcaggcctaagccaggcctcgcagattctggtacaggatgccgaccagaaccgcctggaccgtgacgtgttcaacgaagcctatttgatgcacacctccaccagcccgcaatattcaattattgccagctgcgacgtcgctgctgccatgatggaagcccctggtggcaccgccctggtagaagaatccctcaaagaagcgttggacttccgccgcgccatgcgcaaggtcgacgaagaatggggcacagactggtggtttaaagtctggggtccaactgacctgtccgaagacggcctggaagaacgtgacgcgtggatgctcaaagccaatgaacgctggcatggcttcggcaacctggccgaaggctttaacatgctggatccgatcaaagccaccatcatcaccccaggactagacgtagaaggcgacttttccgatgaattcggcatccccgctgccattgtcaccaagtacctggctgaacacggtgtgatcgttgaaaaaaccggtttatactcattctttatcatgttcaccatcggcattaccaaaggccgctggaacacgatggtggccgcgttacaacaatttaaagacgactacgacaagaatcagccgctgtggaaagtgctgcctgagtttgtacagaaacatccacgctatgaacgcgtaggcctgaaagatctatgcacgcagattcatgaagtttacaaagctaacgacgtagcacgcctgaccacagaaatgtacctgtctgacatggtgccagccatgaaaccgaccgatgctttctcaaaaatggcgcatcgcaaaattgaacgcgtagccattgatgacctcgaaggccgcgtcactgcagtgctgttaacgccctatccgccaggcatcccgttgctgatccctggcgaacgctttaacaaagtcattgtgaactacctcaagtttgcgcgcgagtttaatgagaaattcccaggctttgagacggataaccatggattagtgaagcaaatagtcgatggtaaagccgtgtattatgtggattgcgtgaagcaagaagattaaatttttagtttcactcagcagtttttctactgag

SEQ ID NO: 4 - protein, 748 aa, Methylophilus methylotrophus
MKFRFPIVIIDEDFRSENSSGLGIRVLAKAIEDEGLEVLGVTSYGDLTSFAQQQSRASAFILSIDDEEIVEEKPEAIEQLRNFVQEIRYRNEEIPIFLHGETRTSRHIPNDVLRELHGFIHMNEDTPEFVARLIIREAKAYLDSLPPPFFKALTHYAADGSYSWHCPGHSGGVAFLKSPVGQMFHQFFGENMLRADVCNAVDELGQLLDHTGPVAASERNAARIYNCDHLYFVTNGTSTSNKIVWNSTVAPGDIVVVDRNCHKSVLHSIIMTGAVPVFLMPTRNHFGIIGPIPKSEFAWENIQKKIARNPFATDKNAKPRVLTITQSTYDGVLYNVEEIKEMLDGKIDTLHFDEAWLPHATFHDFYGDYHAIGADRPRCKESMVFSTQSTHKLLAGLSQASQILVQDADQNRLDRDVFNEAYLMHTSTSPQYSIIASCDVAAAMMEAPGGTALVEESLKEALDFRRAMRKVDEEWGTDWWFKVWGPTDLSEDGLEERDAWMLKANERWHGFGNLAEGFNMLDPIKATIITPGLDVEGDFSDEFGIPAAIVTKYLAEHGVIVEKTGLYSFFIMFTIGITKGRWNTMVAALQQFKDDYDKNQPLWKVLPEFVQKHPRYERVGLKDLCTQIHEVYKANDVARLTTEMYLSDMVPAMKPTDAFSKMAHRKIERVAIDDLEGRVTAVLLTPYPPGIPLLIPGERFNKVIVNYLKFAREFNEKFPGFETDNHGLVKQIVDGKAVYYVDCVKQED

SEQ ID NO: 5 - DNA, 30 bp, Artificial Sequence
  Description of Artificial Sequence primer
aaggctgtgcgcaagtgctgttctttgagt

SEQ ID NO: 6 - DNA, 64 bp, Artificial Sequence
  Description of Artificial Sequence primer
ccagcctacacaatcgctcaagacgtgtaatgcacgcatggtagtcaccataaaagtcatggaa

SEQ ID NO: 7 - DNA, 64 bp, Artificial Sequence
  Description of Artificial Sequence primer
ggctaattcccatgtcagccgttaagtgttccatgaactacctcaagtttgcgcgcgagtttaa

SEQ ID NO: 8 - DNA, 30 bp, Artificial Sequence
  Description of Artificial Sequence primer
ggttggtatcagtgtagacacggttgcaag

SEQ ID NO: 9 - DNA, 30 bp, Artificial Sequence
  Description of Artificial Sequence primer
gcattacacgtcttgagcgattgtgtaggc

SEQ ID NO: 10 - DNA, 32 bp, Artificial Sequence
  Description of Artificial Sequence primer
ggaacacttaacggctgacatgggaattagcc

SEQ ID NO: 11 - DNA, 30 bp, Artificial Sequence
  Description of Artificial Sequence primer
aacctgacattcaccaagggcacggtgcaa

SEQ ID NO: 12 - DNA, 30 bp, Artificial Sequence
  Description of Artificial Sequence primer
tttgcgcaaaagcatcgattatccttcccc

SEQ ID NO: 13 - DNA, 33 bp, Artificial Sequence
  Description of Artificial Sequence primer
gccctgcaggagcgcgagtgactggatatcgga

SEQ ID NO: 14 - DNA, 32 bp, Artificial Sequence
  Description of Artificial Sequence primer
gccctgcaggctgtataaatagcaaaggcaac

SEQ ID NO: 15 - DNA, 35 bp, Artificial Sequence
  Description of Artificial Sequence primer
gcctgcagtaaggaaggattttccaggaggaacac

SEQ ID NO: 16 - DNA, 38 bp, Artificial Sequence
  Description of Artificial Sequence primer
gcctgcagaagctttgctcaccgcataatccgtcgcaa

SEQ ID NO: 17 - DNA, 39 bp, Artificial Sequence
  Description of Artificial Sequence primer
agggaattccccgttctggataatgttttttgcgccgac

SEQ ID NO: 18 - DNA, 58 bp, Artificial Sequence
  Description of Artificial Sequence primer
cggatgcatctagagttaacctgcagggtgaaattgttatccgctcacaattccacac

SEQ ID NO: 19 - DNA, 64 bp, Artificial Sequence
  Description of Artificial Sequence primer
catttcctgcaggcaaaggagatgagcgtaatggtgatcatggaaatcttcattacaggtctgc

SEQ ID NO: 20 - DNA, 50 bp, Artificial Sequence
  Description of Artificial Sequence primer
gggcgagctagaagagctccaaaacccgcgaaaactaacccatcaacatc

SEQ ID NO: 21 - DNA, 711 bp, Brevibacterium lactofermentum
  CDS (1)..(711)
atggtgatcatggaaatcttcattacaggtctgcttttgggggccagtcttttactgtccatcggaccgcagaatgtactggtgattaaacaaggaattaagcgcgaaggactcattgcggttcttctcgtgtgtttaatttctgacgtctttttgttcatcgccggcaccttgggcgttgatcttttgtccaatgccgcgccgatcgtgctcgatattatgcgctggggtggcatcgcttacctgttatggtttgccgtcatggcagcgaaagacgccatgacaaacaaggtggaagcgccacagatcattgaagaaacagaaccaaccgtgcccgatgacacgcctttgggcggttcggcggtggccactgacacgcgcaaccgggtgcgggtggaggtgagcgtcgataagcagcgggtttgggtaaagcccatgttgatggcaatcgtgctgacctggttgaacccgaatgcgtatttggacgcgtttgtgtttatcggcggcgtcggcgcgcaatacggcgacaccggacggtggattttcgccgctggcgcgttcgcggcaagcctgatctggttcccgctggtgggtttcggcgcagcagcattgtcacgcccgctgtccagccccaaggtgtggcgctggatcaacgtcgtcgtggcagttgtgatgaccgcattggccatcaaactgatgttgatgggttag

SEQ ID NO: 22 - protein, 236 aa, Brevibacterium lactofermentum
MVIMEIFITGLLLGASLLLSIGPQNVLVIKQGIKREGLIAVLLVCLISDVFLFIAGTLGVDLLSNAAPIVLDIMRWGGIAYLLWFAVMAAKDAMTNKVEAPQIIEETEPTVPDDTPLGGSAVATDTRNRVRVEVSVDKQRVWVKPMLMAIVLTWLNPNAYLDAFVFIGGVGAQYGDTGRWIFAAGAFAASLIWFPLVGFGAAALSRPLSSPKVWRWINVVVAVVMTALAIKLMLMG

SEQ ID NO: 23 - DNA, 712 bp, Brevibacterium lactofermentum
  CDS (1)..(375)
atggtgatcatggaaatcttcattacaggtctgcttttgggggccagtcttttgctgtccatcggaccgcagaatgtactggtgattaaacaaggaattaagcgcgaaggactcattgcggttcttctcgtgtgtttaatttctgacgtctttttgttcatcgccggcaccttgggcgttgatcttttgtccaatgccgcgccgatcgtgctcgatattatgcgctggggtggcatcgcttacctgttatggtttgccgtcatggcagcgaaagacgccatgacaaacaaggtggaagcgccacagatcattgaagaaacagaaccaaccgtgcccgatgacacgcctttgggcgtgttcggcggtggccactgacacgcgcaaccgggtgcgggtggaggtgagcgtcgataagcagcgggtttgggtgaagcccatgttgatggcaatcgtgctgacctggttgaacccgaatgcgtatttggacgcgtttgtgtttatcggcggcgtcggcgcgcaatacggcgacaccggacggtggattttcgccgctggcgcgttcgcggcaagcctgatctggttcccgctggtgggtttcggcgcagcagcattgtcacgcccgctgtccagccccaaggtgtggcgctggatcaacgtcgtcgtggcagttgtgatgaccgcattggccatcaaactgatgttgatgggttag

SEQ ID NO: 24 - protein, 124 aa, Brevibacterium lactofermentum
MVIMEIFITGLLLGASLLLSIGPQNVLVIKQGIKREGLIAVLLVCLISDVFLFIAGTLGVDLLSNAAPIVLDIMRWGGIAYLLWFAVMAAKDAMTNKVEAPQIIEETEPTVPDDTPLGVFGGGH

SEQ ID NO: 25 - DNA, 35 bp, Artificial Sequence
  Description of Artificial Sequence primer
tgacctgcaggtttgcacagaggatggcccatgtt

SEQ ID NO: 26 - DNA, 36 bp, Artificial Sequence
  Description of Artificial Sequence primer
cattctagatccctaaactttacagcaaaccggcat

(19) United States
(12) Patent Application Publication
(10) Pub. No.: US 2004/0229311 A1
(43) Pub. Date: Nov. 18, 2004

(54) Novel lysine decarboxylase gene and method for producing L-lysine

(76) Inventors: Seiko Hirano, Kawasaki-shi (JP); Hisashi Yasueda, Kawasaki-shi (JP)

Correspondence Address: AJINOMOTO CORPORATE SERVICES, LLC, INTELLECTUAL PROPERTY DEPARTMENT, 1120 CONNECTICUT AVE., N.W., WASHINGTON, DC, 20036, US

(21) Appl. No.: 10/784,986

(22) Filed: Feb. 25, 2004

(30) Foreign Application Priority Data
Feb. 25, 2003 (JP) 2003-47185

(51) Int. Cl. C12P 13/08; C07H 21/04; C12N 9/88; C12N 1/21; C12N 15/74

(52) U.S. Cl. 435/69.1; 435/115; 435/232; 435/252.33; 435/320.1; 536/23.2

(57) ABSTRACT

A Methylophilus bacterium in which a gene having a nucleotide sequence identical to a DNA cording for a protein defined in the following (A) or (B) or a gene having homology to the DNA in such a degree that homologous recombination with the DNA occurs is disrupted, thereby expression of the gene is suppressed and the intracellular lysine decarboxylase activity is reduced or eliminated is cultured in a medium containing methanol as a major carbon source to produce and accumulate L-lysine in culture and the L-lysine is collected from the culture: (A) a protein which has the amino acid sequence of SEQ ID NO: 4; (B) a protein which has the amino acid sequence of SEQ ID NO: 4 including substitution, deletion, insertion or addition of one or several amino acid residues and has a lysine decarboxylase activity.

### BACKGROUND OF THE INVENTION

1. Field of the Invention

The present invention relates to a novel lysine decarboxylase gene of Methylophilus bacterium, which is involved in decomposition of L-lysine. The present invention also relates to a Methylophilus bacterium in which expression of the above described gene is suppressed and a method for producing L-lysine using the bacterium.

2. Brief Description of the Related Art

Lysine decarboxylase is an enzyme which catalyzes the reaction generating cadaverine by decarboxylation of L-lysine. For example, in Escherichia coli (E. coli), there are two enzymes designated CadA and Ldc (WO96/17930). Furthermore, based on gene sequence information of genomes or experimental results, it has been suggested that lysine decarboxylase is present in various bacteria including Bacillus halodulans, Bacillus subtilis, Vibrio cholerae, Salmonella typhimurium, Selenomonas ruminantium, Nicotiana glutinosa and so forth (KEGG Database (Release 25.0, January 2003), Y. Takatsuka, et al., Journal of Bacteriology, vol. 182, pp.6732-6741 (2000), Y.-S. Lee and Y.-D. Cho, The Biochemical Journal, vol. 360, pp.657-665 (2001)). However, existence of the enzyme has been uncertain in methanol-utilizing bacteria.

Meanwhile, a method for producing L-lysine using a Methylophilus bacterium is known, and comprises culturing a mutant strain resistant to a lysine analogue such as AEC (S-(2-aminoethyl)-L-cysteine) or a recombinant strain harboring a vector having DNA carrying genetic information involved in the L-lysine biosynthesis (WO00/61723). However, a gene encoding lysine decarboxylase derived from Methylophilus bacteria is not known, and there have been no reports about L-lysine production utilizing a Methylophilus bacterium in which expression of such a gene is suppressed or eliminated.

### SUMMARY OF THE INVENTION

An object of the present ienvention is to obtain a lysine decarboxylase gene of Methylophilus methylotrophus which is a methanol-utilizing bacterium, and to utilize such a gene to create an L-lysine producing bacterium belonging to the genus Methylophilus in which expression of the lysine decarboxylase gene in the cell is suppressed. It is a further object to provide a method for producing L-lysine by culturing such a Methylophilus bacterium.

It is an object of the present invention to provide a protein selected from the group consisting of:

(A) a protein which has the amino acid sequence of SEQ ID NO: 4;

(B) a protein which has the amino acid sequence of SEQ ID NO: 4 including substitution, deletion, insertion or addition of one or several amino acid residues and has a lysine decarboxylase activity.

It is a further object of the present invention to provide a DNA encoding the protein as described above.

It is a further object of the present invention to provide the DNA as described above, which DNA is selected from the group consisting of:

(a) a DNA which has the nucleotide sequence of the nucleotide numbers 684 to 2930 in SEQ ID NO: 3;

(b) a DNA which is hybridizable with a DNA having the nucleotide sequence of the nucleotide numbers 684 to 2930 in SEQ ID NO: 3 under stringent conditions, and codes for a protein having lysine decarboxylase activity.

It is a still further object of the present invention to provide the DNA as described above, which is derived from a chromosome of a Methylophilus bacterium.

It is even a further object of the present invention to provide a Methylophilus bacterium, which has an ability to produce L-lysine and is modified so that intracellular lysine decarboxylase activity is reduced or eliminated.

It is a further object ofthe present invention to provide the Methylophilus bacterium as described above, wherein a gene on a chromosome having a nucleotide sequence identical to the DNA as described above is disrupted or a gene on a chromosome having homology to the DNA as described above to such a degree that homologous recombination with the DNA occurs is disrupted, thereby expression of the gene is suppressed and the intracellular lysine decarboxylase activity is reduced or eliminated.

It is a further object of the present invention to provide a method for producing L-lysine, comprising the steps of culturing the Methylophilus bacterium as described above in a medium containing methanol as a major carbon source to produce and accumulate L-lysine in culture and collecting the L-lysine from the culture.

According to the present invention, it becomes possible to provide a novel lysine decarboxylase and a gene encoding the enzyme. Furthermore, by culturing a Methylophilus bacterium which has an ability to produce L-lysine and in which expression of the gene is suppressed, L-lysine can be efficiently produced.

### DETAILED DESCRIPTION OF THE INVENTION

The inventors ofthe present invention conducted research to determine whether lysine decarboxylase existed in Methylophilus bacteria, and as a result, they found an open reading frame (henceforth abbreviated as “orf”) having homology to a known lysine decarboxylase gene derived from a DNA sequence on the genome of Methylophilus methylotrophus. As for the homology of the amino acid sequence encoded by the gene, homology (rate of the same amino acids) of 38.18% to the cadA product of Escherichia coli (E. coli K12, NCBI: AAC77092) and homology of 37.85% to the ldcC product of the same (E. coli K12, NCBI: AAC73297) was found. Moreover, the amino acid sequence encoded by the orf also had about 38.11 % homology to arginine decarboxylase, which is the gene product of adiA of Escherichia coli (E. coli K12, NCBI: AAC77078), and thus the new Idc gene was identified.

Therefore, the present inventors attempted to disrupt the above described orf of Methylophilus methylotrophus to investigate its function. As a result, the obtained strain no longer grew in the SEII medium, whereby usually a wild-type strain of Methylophilus methylotrophus is able to grow. This was an unexpected result, because Escherichia coli and so forth do not show any particular auxotrophy even if cadA and ldcC are deleted.

0017 Since it was considered that there was a nutrient that became essential for Methylophilus methylotrophus due to the deficiency of the orf and was not contained in the components of the SEII medium, cadaverine, which is a degradation product of L-lysine, or agmatine, which is a degradation product of L-arginine, was added to the medium in an appropriate amount. As a result, the strain deficient in the orf was able to grow in the medium.

Therefore, it was found that, in Methylophilus methylotrophus, the protein encoded by that orf was essential for growth in a typical minimal medium, and cadaverine or agmatine was necessary for growth of a strain deficient in that orf. Based on the above, the gene containing this orf was designated an ldc gene.

Furthermore, when expression of the ldc gene was suppressed in an L-lysine-producing strain which was bred from Methylophilus methylotrophus, the L-lysine production was improved, and thus the present invention was accomplished.

Hereafter, the present invention will be explained in detail.

Lysine decarboxylase of the present invention and DNA encoding it

The lysine decarboxylase of the present invention is a protein defined in the following (A) or (B):

(A) a protein which has the amino acid sequence of SEQ ID NO: 4;

(B) a protein which has the amino acid sequence of SEQ ID NO: 4 including substitution, deletion, insertion or addition of one or several amino acid residues and has a lysine decarboxylase activity.

The DNA of the present invention encodes the protein defined in the above (A) or (B).

The DNA of the present invention (henceforth also referred to as the “ldc gene”) can be isolated and obtained from a chromosomal DNA of a Methylophilus bacterium, for example, Methylophilus methylotrophus. A wild-type strain of Methylophilus methylotrophus, the AS1 strain (NCIMB No. 10515), is available form the National Collections of Industrial and Marine Bacteria (Address: NCIMB Lts., Torry Research Station, 135, Abbey Road, Aberdeen AB9 8DG, United Kingdom). Although a typical culture method for this strain is described in the catalogue of NCIMB, it can also be grown in the SEII medium described in the examples sections.

The genomic DNA of the AS1 strain can be prepared by a known method, and a commercially available kit for preparing genome may be used.

The DNA of the present invention can be obtained by synthesizing primers based on the nucleotide sequence of the nucleotide numbers 684 to 2930 in SEQ ID NO: 3 and then amplifying the DNA by PCR (polymerase chain reaction) using a chromosomal DNA of a bacterium such as Methylophilus bacterium as a template.

Furthermore, the DNA of the present invention can also be obtained by colony hybridization using a probe prepared based on the aforementioned nucleotide sequence or a partial fragment amplified by PCR as a probe.

Preparation techniques of the genomic DNA library, hybridization, PCR, preparation of plasmid DNA, digestion and ligation of DNA, transformation and so forth used for cloning of the DNA of the present invention are described in Sambrook, J., Fritsch, E. F., Maniatis, T., Molecular Cloning, Cold Spring Harbor Laboratory Press, Third Edition (2001).

Examples of the primers used for the aforementioned PCR include, but are not limited to, oligonucleotides of SEQ ID NOS: 1 and 2.

The nucleotide sequence of the ldc gene isolated from the genome of Methylophilus methylotrophus, which was obtained as described above, is shown as SEQ ID NO: 3. Furthermore, the amino acid sequence of lysine decarboxylase encoded thereby is shown as SEQ ID NO: 4.

As for the aforementioned amino acid sequence, a known database was searched for amino acid sequences having homology thereto. As a result, two kinds of lysine decarboxylases (encoded by cadA and ldcC) and arginine decarboxylase (encoded by adiA) of Escherichia coli had homologies of 38.18%, 37.85% and 38.11%, respectively, to the aforementioned amino acid sequence. The homologies were calculated as ratios of the same amino acid residues to the total number of amino acid residues of the regions used for comparison.

The DNA of the present invention may code for an amino acid sequence including substitution, deletion, insertion or addition of one or several amino acid residues at one or more positions, so long as the activity of the encoded lysine decarboxylase is not substantially degraded. The term “several” as used herein varies depending on the positions of the amino acid residues in the three-dimensional structures of the protein and the types of amino acid. However, the amino acid sequence may be a sequence exhibiting 70% or more, preferably 80% or more, more preferably 90% or more, of homology to the whole amino acid sequence constituting the lysine decarboxylase and having the activity of lysine decarboxylase. Specifically, “several” is preferably between 2 to 20, more preferably between 2 to 10. The aforementioned activity of lysine decarboxylase means an activity for catalyzing the reaction producing cadaverine by decarboxylation of L-lysine.

A DNA encoding a protein substantially identical to the aforementioned lysine decarboxylase can be obtained by modifying the nucleotide sequence shown in SEQ ID NO: 3. For example, site-specific mutagenesis can be employed so that substitution, deletion, insertion or addition of an amino acid residue or residues occurs at a specific site. Furthermore, a DNA modified as described above can also be obtained by conventionally-known mutation treatments. Examples of such mutation treatments include a method of treating the ldc gene in vitro with hydroxylamine or the like, and a method of treating a microorganism, for example, an Escherichia bacterium, containing ldc gene with ultraviolet ray irradiation or a mutagenesis agent used in a usual mutation treatment such as N-methyl-N′-nitro-N-nitrosoguanidine (NTG) or EMS.

The substitution, deletion, insertion, addition, inversion or the like of nucleotides described above also includes a naturally occurring mutation on the basis of, for example, individual difference or difference in species of microorganisms that contain the ldc gene.

A DNA encoding the substantially same protein as lysine decarboxylase can be obtained by expressing such a DNA having a mutation as described above in a suitable cell and examining the activity of expressed lysine decarboxylase. A DNA encoding substantially the same protein as lysine decarboxylase can also be obtained by isolating a DNA hybridizable with a DNA having the nucleotide sequence corresponding to nucleotide numbers of 684 to 2930 of the nucleotide sequence shown in SEQ ID NO: 3 or a probe that can be prepared from the nucleotide sequence under stringent conditions and encoding a protein having the activity of lysine decarboxylase from a cell harboring the ldc gene having a mutation.

The “stringent conditions” include conditions under which a so-called specific hybrid is formed, and non-specific hybrid is not formed. It is difficult to clearly express this condition by using any numerical value. However, for example, the stringent conditions include a condition under which DNAs having high homology, for example, DNAs having homology of 70% or more, preferably 80% or more, more preferably 90% or more, most preferably 95% or more hybridized with each other, and DNAs having homology lower than the above do not hybridize with each other. Alternatively, the stringent conditions include a condition whereby DNAs hybridize with each other at a salt concentration corresponding to typical washing condition of Southern hybridization, i.e., 1×SSC, 0.1% SDS, preferably 0.1×SSC, 0.1% SDS, at 60° C.

A partial sequence of the ldc gene can also be used as the probe. Such a probe can be produced by PCR using oligonucleotides prepared based on the nucleotide sequence of the gene as primers and a DNA fragment containing the gene as a template using methods well known to those skilled in the art. When a DNA fragment in a length of about 300 bp is used as the probe, the washing condition of hybridization can be, for example, 50° C., 2×SSC and 0.1% SDS.

The activity of lysine decarboxylase can be measured by the method described in Y.-S. Lee and Y.-D. Cho, The Biochemical Journal, vol. 360, pp.657-665 (2001).

The ldc gene of the present invention can be used for, in addition to the construction of an ldc gene-disrupted strain as described later, for example, production of the lysine decarboxylase of the present invention. That is, the lysine decarboxylase can be produced by introducing the ldc gene into a suitable host microorganism to allow expression of the gene. This can be performed in the same manner as a usual method used for production of a useful protein utilizing gene recombination techniques. That is, a DNA encoding lysine decarboxylase can be inserted into a vector including a suitable promoter, a host such as Escherichia coli can be transformed with the obtained recombinant vector, and the transformant can be cultured to allow expression of the aforementioned gene. Examples of the host include, but are not limited to, Escherichia coli, Bacillus subtilis,yeast and so forth. The promoter may be any promoter that functions in the host used, and examples include lac, trp, tac, trc, recA, T7 (Lecture of Biochemical Experiments, New Edition, vol. 1, Protein, VI Synthesis and Expression, edited by the Japanese Biochemical Society, p.166, Yasueda, Matsui, 1992, published by Tokyo Kagaku Dojin), PGK, ADH1, GPD, MFα1, SUC2, PHO5, GAL1, GAL4 (Lecture of Biochemical Experiments, New Edition, vol. 1, Protein, VI Synthesis and Expression, edited by the Japanese Biochemical Society, p.215, Sakai et al., 1992, published by Tokyo Kagaku Dojin) and so forth.

The lysine decarboxylase can be collected from a host microorganism in the same manner as that used for production of a usual recombinant protein.

Methylophilus bacterium of the present invention

The bacterium of the present invention is a Methylophilus bacterium having an ability to produce L-lysine and modified so that the intracellular lysine decarboxylase activity is reduced or eliminated.

An example of the Methylophilus bacterium includes Methylophilus methylotrophus. The “ability to produce L-lysine” referred to in the present invention means an ability of the bacterium of the present invention to cause accumulatation of a significant amount of L-lysine in a medium when the bacterium is cultured in the medium.

The reduction or elimination of the intracellular lysine decarboxylase activity is attained by, for example, suppressing expression of the ldc gene. The reduction or elimination of the intracellular lysine decarboxylase activity can also be attained by modifying the structure of the lysine decarboxylase enzyme encoded by the gene to reduce or eliminate the specific activity of the lysine decarboxylase. Examples of the method for obtaining such a Methylophilus bacterium in which the intracellular lysine decarboxylase activity is reduced or eliminated include a method of treating a Methylophilus bacterium with ultraviolet ray irradiation or a mutagenesis agent used in a usual mutagenesis treatment such as N-methyl-N′-nitro-N-nitrosoguanidine (NTG) or EMS and selecting a mutant strain showing reduced activity of lysine decarboxylase.

A preferred embodiment of the bacterium of the present invention is a Methylophilus bacterium in which the ldc gene on a chromosome is disrupted, thereby expression of the gene is suppressed and the intracellular lysine decarboxylase activity is reduced or eliminated. The ldc gene referred to in this embodiment include a gene encoding lysine decarboxylase having the amino acid sequence of SEQ ID NO: 4 and a gene having homology to the gene to such a degree that homologous recombination occurs with the gene having the amino acid sequence of SEQ ID NO: 4. The aforementioned homology to such a degree that homologous recombination occurs is preferably homology of 90% or more, more preferably 95% or more, particularly preferably 99% or more.

The ldc gene on a chromosome can be disrupted by a method based on gene substitution utilizing homologous recombination (Experiments in Molecular Genetics, Cold Spring Harbor Laboratory Press (1972); Matsuyama, S. & Mizushima, S., J. Bacteriol., 162, 1196 (1985)) as described in the examples sections. The ability to cause homologous recombination is a property generally possessed by bacteria, and the inventors of the present invention found that gene substitution utilizing homologous recombination was also possible in Methylophilus bacteria. Specifically, a Methylophilus bacterium is transformed with a DNA containing the ldc gene modified so as not to produce lysine decarboxylase that normally functions (deletion-type ldc gene), and recombination is caused between the deletion-type ldc gene and the ldc gene on a chromosome. Thereafter, if recombination occurs again at a site on the chromosome to which the plasmid is incorporated, the plasmid is eliminated from the chromosome. At this time, depending on the site where the recombination occurs, the deletion-type gene may be fixed on the chromosome, and the native gene may be eliminated from the chromosome along with the plasmid, or the native gene may be fixed on the chromosome, and the deletion-type gene may be eliminated from the chromosome along with the plasmid. By selecting such a strain in which the former occurred, a strain in which the deletion-type gene is substituted for the native gene on the chromosome can be obtained.

Furthermore, the inventors of the present invention also found that, in Methylophilus methylotrophus, introduction of a gene homologous to a desired gene on a chromosome in the form of a linear DNA fragment caused homologous recombination between the desired gene on the chromosome and the homologous gene on the introduced linear DNA fragment in the cell, and thereby gene substitution could be attained, and such a technique can also be applied. An example of gene substitution performed by using this technique is described in the examples sections.

Examples of the aforementioned deletion-type ldc gene include genes in which substitution, deletion, insertion, addition or inversion of one or more nucleotides is caused in the nucleotide sequence of coding region and thereby specific activity of the encoded protein is reduced or eliminated as well as genes of which internal portion or end portion of the coding region is deleted, genes of which coding region is inserted with another sequence and so forth. Examples of other sequences include marker genes such as the kanamycin resistance gene.

Expression of the ldc gene on a chromosome can also be reduced or eliminated by introducing substitution, deletion, insertion, addition or inversion of one or several nucleotides into a promoter sequence of the gene to reduce the promoter activity and thereby suppressing expression of the gene at a transcription level (see Rosenberg, M. & Court, D., Ann. Rev. Genetics, 13, p.319 (1979); Youderian, P., Bouvier, S. & Susskind, M., Cell, 30, pp.843-853 (1982)).

Furthermore, expression of the ldc gene can also be suppressed at a translation level by introducing substitution, deletion, insertion, addition or inversion of one or several nucleotides into a region between the SD sequence and the initiation codon of the gene (see Dunn, J. J., Buzash-Pollert, E. & Studier, F. W., Proc. Natl. Acad. Sci. U.S.A., 75, p.2743 (1978)).

The modification of a promoter or a region between the SD sequence and the initiation codon described above can be performed in the same manner as that for the aforementioned gene substitution. Site-specific mutagenesis (Kramer, W. & Frits, H. J., Methods in Enzymology, 154, 350 (1987)) and use of a treatment with a chemical agent such as sodium hyposulfite or hydroxylamine (Shortle, D. and Nathans, D., Proc. Natl. Acad. Sci. U.S.A., 75, 270 (1978)) can be specifically employed in order to introduce substitution, deletion, insertion, addition or inversion of nucleotides into a gene.

Site-specific mutagenesis is a method using synthetic oligonucleotides, which can introduce arbitrary substitution, deletion, insertion, addition or inversion into specific base pairs. In order to utilize this method, a plasmid harboring a desired gene that is cloned and has a known DNA nucleotide sequence is first denatured to prepare a single strand. Then, a synthetic oligonucleotide complementary to a region where a mutation is desired to be introduced is synthesized. In this synthesis, the sequence of the synthetic oligonucleotide is not prepared as a completely complementary sequence, but is made to include substitution, deletion, insertion, addition or inversion of an arbitrary nucleotide or nucleotides. Thereafter, the single-stranded DNA and the synthetic oligonucleotide including substitution, deletion, insertion, addition or inversion of an arbitrary nucleotide or nucleotides are annealed, and a complete double-stranded plasmid is synthesized using Klenow fragment of DNA polymerase I and T4 ligase and introduced into competent cells of Escherichia coli. Some of the transformants obtained as described above would have a plasmid containing the gene in which substitution, deletion, insertion, addition or inversion of an arbitrary nucleotide or nucleotides is fixed. A similar method that enables introduction of mutation into a desired gene and thereby enables modification or disruption of the gene includes the recombinant PCR method (PCR Technology, Stockton Press (1989)).

By replacing the native gene on a chromosome of a Methylophilus bacterium with the gene introduced with a mutation and thereby modified or disrupted as described above, expression of the ldc gene in the cell can be suppressed.

The Methylophilus bacterium having reduced or eliminated lysine decarboxylase activity is a Methylophilus bacterium having an ability to produce L-lysine. A Methylophilus bacterium having an ability to produce L-lysine, for example, a Methylophilus methylotrophus strain, can be obtained by subjecting such a strain which does not have an ability to produce L-lysine or has a low ability to produce L-lysine to a mutagenesis treatment to impart to it resistance to an L-lysine analogue such as S-(2-aminoethyl)-L-cysteine (hereinafter referred to as “AEC”). Examples of the method for the mutagenesis treatment include, but are not limited to, methods of treating cells of Escherichia coli with a chemical mutagenesis agent such as NTG or EMS or with an ultraviolet ray, radiation exposure or the like. Specific examples of such a strain include Methylophilus methylotrophus AJ13608. This strain was bred by imparting the AEC resistance to the Methylophilus methylotrophus AS1 strain. The Methylophilus methylotrophus AJ13608 was deposited at the National Institute of Bioscience and Human-Technology, Agency of Industrial Science and Technology (currently, the independent administrative agency, National Institute of Advanced Industrial Science and Technology, International Patent Organism Depositary, Tsukuba Central 6, 1-1, Higashi 1-Chome, Tsukuba-shi, Ibaraki-ken, 305-8566, Japan) on Jun. 10, 1999 and received an accession number of FERM P-17416. Then, the deposit was converted to an international deposit under the provisions of the Budapest Treaty on Mar. 31, 2000 and received an accession number of FERM BP-7112.

A Methylophilus methylotrophus having an ability to produce L-lysine can also be bred by introducing a DNA carrying genetic information involved in the biosynthesis of L-lysine or enhancing the expression of the DNA with a genetic recombination technique. The gene or genes to be introduced encodes an enzyme of the biosynthetic pathway of L-lysine such as dihydrodipicolinate synthase and succinyl diaminopimelate transaminase. In the case of a gene of enzyme suffering from feedback inhibition by L-lysine such as dihydrodipicolinate synthase, it is preferable to use a mutant gene encoding the enzyme for which inhibition is desensitized.

Furthermore, an ability to produce L-lysine can also be improved by enhancing an activity of a protein involved in secretion of L-lysine. For example, as a protein involved in secretion of L-lysine, the LysE protein encoded by the lysE gene is known (M. Vrljic, H. Sahm and L. Eggeling, Molecular Microbiology 22, pp.815-826 (1996); International Patent Publication WO97/23597). The inventors of the present invention confirmed that, although a wild-type lysE derived form Brevibacterium bacteria did not function at all in Methylophilus bacteria, it could be modified to function in Methylophilus bacteria. Examples of such variants of the LysE protein include LysE24 described in the examples sections (see US-2003-0124687-A1).

The LysE protein that is encoded by the lysE gene has six hydrophobic helix regions. Some of these hydrophobic regions are estimated to be transmembrane domains. It is also estimated that a region between the third and fourth regions relative to the N-terminus is hydrophilic and has a loop structure. In the present invention, this hydrophilic region is called a loop region. The nucleotide sequence of wild-type lysE and the amino acid sequence of the LysE protein of Brevibacterium lactofermentum are shown in SEQ ID NOS: 21 and 22. In this amino acid sequence, the hydrophobic helix regions correspond to the amino acid numbers 5-20, 37-58, 67-93, 146-168, 181-203 and 211-232. The loop region corresponds to the amino acid numbers 94 to 145.

The inventors of the present invention found that the lysE gene was lethal in Methylophilus bacteria, but that a DNA encoding a variant of the LysE protein that did not have the loop region or substantially consisted of only the hydrophobic helixes increased the secretion of L-lysine to the outside of cells of methanol-utilizing bacterium (US-2003-0124687-A1). The lysE24 encodes such a mutant LysE protein lacking the aforementioned loop region that is contained in a wild-type LysE protein or that substantially consists of only the hydrophobic helixes.

The aforementioned mutant LysE is not particularly limited so long as it has one or more hydrophobic helixes and, when expressed in a methanol-utilizing bacterium, results in increased secretion of L-lysine. Specifically, a DNA coding for a mutant LysE that has all of the first to sixth hydrophobic helixes relative to the N-terminus is encompassed. More specifically, a DNA encoding a peptide containing the first to third hydrophobic helixes relative to the N-terminus, and encoding a peptide containing the fourth to sixth hydrophobic helixes relative to the N-terminus is encompassed. The aforementioned lysE24 is an example of the mutant lysE that encodes a peptide containing the first to third hydrophobic helixes and a peptide containing the fourth to sixth hydrophobic helixes. The lysE24 gene is introduced by a mutation with a stop codon downstream from the region encoding the third hydrophobic helix. The inventors of the present invention confirmed that, if a region downstream from this stop codon was deleted, the mutant lysE24 gene did not cause L-lysine to accumulate in the medium when expressed in Methylophilus methylotrophus AS1 strain. Therefore, it is estimated that a peptide containing the first to third hydrophobic helixes and a peptide containing the fourth to sixth hydrophobic helixes are separately translated and function in a Methylophilus bacterium. The results show that introduction of the lysE24 gene into a Methylophilus bacterium will result in improvement of the production of L-lysine.

Any microorganism can be used to generate a DNA encoding a protein involved in secretion of L-lysine to the outside of a cell, i.e., the lysE gene or its homologous gene, so long as it has a variant of the gene that can express the L-lysine secretion activity in a methanol-utilizing bacterium.

Specifically, examples of such microorganisms include, but are not limited to, coryneform bacterium such as Corynebacterium glutamicum and Brevibacterium lactofermentum, Escherichia bacteria such as Escherichia coli, Pseudomonas bacteria such as Pseudomonas aeruginosa, Mycobacterium bacteria such as Mycobacterium tuberculosis and so forth.

In order to enhance expression of the L-lysine secretion gene in a methanol-utilizing bacterium, the gene fragment is ligated to a vector which is able to function in a Methylophilus bacterium, preferably a multi-copy type vector, to prepare recombinant DNA which is then used to transform the methanol-utilizing bacterium host. Alternatively, the gene can be incorporated into a transposon and introduced into a chromosome. Furthermore, a promoter that induces potent transcription in a methanol-utilizing bacterium can be ligated upstream from the gene.

To introduce an objective gene such as an L-lysine byosynthesis gene or L-lysine secretion gene into Methylophilus bacteria and enhance its expression, the gene may be ligated to a vector autonomously replicable in a cell of Methylophilus bacteria to prepare a recombinant DNA, which is then used to transform Methylophilus methylotrophus by, for example, electroporation. In addition, it is also possible to incorporate an objective gene into a host chromosome by a method using transduction, transposon (D. E. Berg, and C. M. Berg, Bio/Technol., 1, p.417 (1983)), Mu phage, (Japanese Patent Laid-open (Kokai) No. 2-109985) or homologous recombination (Experiments in Molecular Genetics, Cold Spring Harbor Lab. (1972)).

The vectors autonomously replicable in Methylophilus bacteria include, but are not limited to, RSF1010, which is a wide host range vector, and derivatives thereof, for example, pAYC32 (Chistorerdov, A. Y., Tsygankov, Y. D., Plasmid, 16, pp.161-167 (1986)) and pMFY42 (Gene, 44, p.53 (1990)), pBBR1 and those derived from derivatives thereof (Kovach, M. E., et al., Gene, 166, pp.175-176 (1995)), pRK310 and those derived from derivatives thereof (Edts. Murrell, J. C., and Dalton, H., Methane and methanol utilizers, Plenum Press, pp.183-206 (1992)) and so forth.

A Methylophilus bacterium which has an ability to produce L-lysine and in which the lysine decarboxylase activity is reduced or eliminated can be obtained by imparting an ability to produce L-lysine to a Methylophilus bacterium in which the lysine decarboxylase activity is reduced or eliminated. Furthermore, such a bacterium as mentioned above can also be obtained by modifying a Methylophilus bacterium having an ability to produce L-lysine so that the lysine decarboxylase activity is reduced or eliminated.

Production of L-lysine

Culturing the Methylophilus bacterium in which the lysine decarboxylase activity is reduced or eliminated obtained as described above in a medium containing methanol as a major carbon source results in production of marked amount of of L-lysine and accumulation of the produced L-lysine in the medium. Thus, utilization of the Methylophilus bacterium of the present invention having an ability to produce L-lysine in which the lysine decarboxylase activity is reduced or eliminated is effective for improvement of accumulating L-lysine.

The medium used for the production of L-lysine is a typical medium that contains a carbon source, nitrogen source, inorganic ions and other organic trace nutrients as required. The major carbon source is methanol. However, sugars such as glucose, lactose, galactose, fructose and starch hydrolysate, alcohols such as glycerol and sorbitol, and organic acids such as fumaric acid, citric acid, succinic acid and pyruvic acid may be used together. The expression “methanol is used as a major carbon source” means that methanol content in the total carbon source is 50% (w/w) or more, preferably 80% (w/w) or more, of the total carbon source. If methanol is used as a carbon source, the concentration thereof is usually between 0.001% to 4% (w/v), preferably 0.1% to 2% (w/v). Furthermore, when glucose etc. is added, the concentration thereof is usually between 0.1% to 3% (w/w), preferably between 0.1% to 1% (w/v).

As the nitrogen source, inorganic ammonium salts such as ammonium sulfate, ammonium chloride and ammonium phosphate, organic nitrogen source such as soybean hydrolysate, ammonia gas, aqueous ammonia and so forth can be used.

As the inorganic ions (or sources thereof), a small amount of potassium phosphate, magnesium sulfate, iron ions, manganese ions and so forth are added to the medium. As the organic trace nutrients, vitamin B₁, yeast extract and so forth may be added to the medium in suitable amounts.

The culture is preferably performed for about 16 to 72 hours under aerobic conditions. The culture temperature is controlled to be between 25° C. to 45° C., and pH is controlled to be between 5 to 8 during the culture. Inorganic or organic acidic or alkaline substances, ammonia gas and so forth can be used to adjust the pH.

After completion of the culture, L-lysine can be collected from a fermentation broth by for, example, typical methods utilizing ion exchange resins, precipitation method and so forth in combination.

### EXAMPLES

Hereafter, the present invention will be explained more specifically with reference to the following non-limiting examples.

### Example 1

Cloning of Lysine Decarboxylase Gene (ldc) of Methylophilus methylotrophus

In order to obtain a chromosomal DNA from the Methylophilus methylotrophus AS1 wild strain, the AS1 strain was inoculated into 50 mL of the SEII medium (composition: 5.0 g/L of (NH₄)₂SO₄, 1.9 g/L of K₂HPO₄, 1.56 g/L of NaH₂PO₄.2H₂O, 200 mg/L of MgSO₄-7H₂O, 72 mg/L of CaCl₂.6H₂O, 5 μg/L of CuSO₄.5H₂O, 25 μg/L of MnSO₄.5H₂O, 23 μg/L of ZnSO₄.7H₂O, 9.7 mg/L of FeCl3-6H₂O, 0.5% (v/v) of methanol) and cultured overnight at 37° C. with shaking. Then, the culture broth was centrifuged to collect the cells. A chromosomal DNA was prepared from the obtained cells by using a commercially available kit (Genomic DNA Purification Kit (produced by Edge Biosystems)) according to the attached operation manual.

The chromosomal DNA was used as a template together with the DNA primers of SEQ ID NOS: 1 and 2 to perform PCR (a cycle consisting of denaturation at 98° C. for 10 seconds, annealing at 55° C. for 30 seconds extension at 72° C. for 3 minutes was repeated for 25 cycles). Pyrobest polymerase (Takara Shuzo) was used. As a result, a DNA fragment having a size of about 3.0 kilo base pairs (henceforth abbreviated as “kbp”) was obtained.

Then, the obtained fragment was sequenced by the method described in Sambrook, J., Fritsch, E. F., Maniatis, T., Molecular Cloning, Cold Spring Harbor Laboratory Press, Third Edition (2001). It became clear that the region from the restriction enzyme EcoRV site to the restriction enzyme DdeI site on the DNA fragment had the nucleotide sequence shown as SEQ ID NO: 3. In this DNA sequence, an open reading frame (henceforth also abbreviated as “orf”) encoding the amino acid sequence shown as SEQ ID NO: 4 was contained. This orf was designated orf#3098. The gene encoding the amino acid sequence shown as SEQ ID NO: 4 was designated the ldc gene.

### Example 2

Preparation of ldc Gene-Disrupted Methylophilus methylotrophus Strain

(1) Preparation of Fragment for Disruption of ldc Gene

The chromosomal DNA obtained in Example 1 was used as a template together with the DNA primers shown in SEQ ID NOS: 5 and 6 to perform PCR (reaction conditions: TaKaRa Ex Taq was used, a cycle consisting of denaturation at 94° C. for 30 seconds, annealing at 60° C. for 30 seconds and DNA strand extension reaction at 72° C. for 2 minutes was repeated for 25 cycles) and thereby obtain a fragment of about 1.3 kb. PCR was also performed by using the primers shown in SEQ ID NOS: 7 and 8 under the same conditions to obtain a DNA fragment having a size of about 2.0 kb.

PCR was also performed by using the plasmid pKD4 (GenBank Accession No. AY048743, Datsenko, K. A. et al., Proc. Natl. Acad. Sci. U.S.A., 97 (12), 6640-6645 (2000)) as a template and the primers shown in SEQ ID NOS: 9 and 10 under the same conditions as mentioned above to obtain a DNA fragment containing a kanamycin resistance (Kmr) gene (about 1.5 kb).

The three kinds of DNA fragments described above were mixed and used as a template together with the primers shown in SEQ ID NOS: 11 and 12 to perform PCR (reaction conditions: TaKaRa Ex Taq was used, a cycle consisting of denaturation at 94° C. for 30 seconds, annealing at 60° C. for 30 seconds and DNA strand extension reaction at 72° C. for 4 minutes and 30 seconds was repeated for 25 cycles) and thereby obtain a fragment of about 4.7 kb. This fragment contained the ldc gene interrupted with the kanamycin resistance gene. This fragment was purified by using a commercially available kit (Wizard PCR Preps DNA Purification System produced by Promega) and then subjected to ethanol precipitation, and the precipitates were dissolved in TE solution (10 mM Tris-HCl (pH 7.5), 1 mM EDTA solution). This DNA solution was used in the following operation as a fragment for gene disruption.

(2) Acquisition of ldc Gene Deficient Strain of Methylophilus methylotrophus

Then, the gene fragment for gene disruption described above was introduced into the Methylophilus methylotrophus AS1 strain. The electroporation method (Canadian Journal of Microbiology, 43, 197 (1997)) was used for the transformation. Specific procedure was as follows.

The Methylophilus methylotrophus ASI strain was cultured in the SEII liquid medium (methanol concentration: 0.5% (v/v)) at 37° C. for 16 hours with shaking, and 20 ml of the culture broth was centrifuged at 10,000 rpm for 10 minutes to collect the cells. The cells were added with 1 mM HEPES buffer (pH 7.2, 20 ml), suspended in it and centrifuged, and this operation was performed twice. Finally, 1 ml of the same buffer was added to the cells to prepare cell suspension and used as electro cells for electroporation. Then, about 1 μg of the aforementioned DNA fragment containing the ldc gene interrupted with the kanamycin resistance gene (ldc::KmR) was added to 100 μl of the electro cells, and electric pulses were applied with the conditions of 18.5 kV/cm, 25 μF and 200 Ω to perform electroporation and thereby introduce the DNA fragment into the cells. The SEII liquid medium was immediately added to this cell suspension, and the cells were cultured at 37° C. for 3 hours.

Then, this culture broth was applied to the SEII agar medium containing 20 μg/ml of kanamycin and incubated at 37° C. After the culture of 48 hours, several tens of colonies emerged on the plate. Among these, 20 strains were randomly selected, and disruption of the objective gene in these strains was confirmed by a detection method based on the PCR method. That is, the aforementioned colonies that appeared were each suspended in 20 μl of sterilized water, added with 5 μl of 1 mg/ml Proteinase K and 25 μl of P solution (solution containing 40 mM Tris, 0.5% Tween 20, 1% Nonidet P-40, 1 mM EDTA (adjusted to pH 8.0 with HCl)), stirred and incubated at 60° C. for 20 minutes and at 95° C. for 5 minutes. This reaction mixture was used as a template together with the primers shown in SEQ ID NOS: 11 and 12 to perform PCR (reaction conditions: TaKaRa Ex Taq was used, a cycle consisting of denaturation at 94° C. for 30 seconds, annealing at 60° C. for 30 seconds and DNA strand extension reaction at 72° C. for 4 minutes and 30 seconds was repeated for 25 cycles) and thereby confirm the disruption of the objective gene. As a result, it was found that 10 strains were the intended gene-disrupted strains. Therefore, one strain among them was designated a DLC10 strain (MLDC strain) and used in the following experiments.

(3) Phenotype of ldc Gene Deficient Strain

The DLC10 strain prepared in the above (2) was a strain selected as a strain that could grow on the SEII agar medium containing kanamycin. However, it was found that it could not continue to grow when it was subcultured on the same agar medium. Therefore, it was investigated whether the growth inhibition could be complemented by addition of cadaverine (CAD) and agmatine (AGM), which are reaction products of lysine decarboxylase (LDC) and arginine decarboxylase (ADC), respectively, to the medium.

A medium consisting of 4 ml of liquid SEII medium containing 20 μg/ml of kanamycin and added with cadaverine or agmatine at a concentration of 1 g/l was prepared. Then, the aforementioned DLC10 strain was inoculated to the medium and cultured at 37° C. with shaking at 116 rpm, and the growth was examined. As a result, it was found that the DLC10 strain could not grow on the medium which lacked cadaverine and agmatine, whereas the strain was able to grow on the medium containing one of these substances. Moreover, the addition of cadaverine showed better growth restoration effect compared with the addition of agmatine.

(4) Confirmation of Complementation of ldc Deficient Strain by Introduction of orf#3098

It was verified whether the cadaverine auxotrophy for growth of the aforementioned ldc deficient strain could be complemented by introduction of orf#3098 obtained in Example 1. First, a plasmid for introducing DNA containing only orf#3098 into the ldc deficent strain was prepared. The chromosomal DNA described in Example 1 was used as a template together with DNA primers having the sequence shown as SEQ ID NOS: 13 and 14 (Sse83871 site was ligated to the 5′ end side) to perform PCR (amplification reaction conditions: Pyrobest DNA polymerase produced by Takara Shuzo was used, a cycle consisting of denaturation at 98° C. for 10 seconds, annealing at 55° C. for 30 seconds and DNA strand extension reaction at 72° C. for 3 minutes was repeated for 25 cycles). The obtained DNA fragment having a size of about 3 kb was digested with the restriction enzyme Sse8387I (Takara Shuzo). This DNA fragment was ligated with the vector pRStac similarly digested with Sse8387I and then subjected to a dephosphorylation treatment (Ligation Kit Ver. 2 produced by Takara Shuzo was used). The plasmid carrying orf#3098 (in the forward direction with respect to the tac promoter) prepared as described above was designated pRS-orf#3098.

pRStac was constructed by introducing the tac promoter into a known plasmid pRS (see International Patent Publication in Japanese (Kohyo) No. 3-501682). pRS is a plasmid having the vector segment of the pVIC40 plasmid (International Patent Publication WO90/04636, International Patent Publication in Japanese No. 3-501682) and obtained from pVIC40 by deleting a DNA region encoding the threonine operon contained in the plasmid. The plasmid pVIC40 is derived from a wide host range vector plasmid pAYC32 (Chistorerdov, A. Y, Tsygankov, Y. D., Plasmid, 1986, 16, 161-167), which is a derivative of RSF1010.

First, the plasmid pRStac having the tac promoter was constructed from pRS. The pRS vector was digested with restriction enzymes EcoRI and PstI and added to a phenol/chloroform solution and mixed to terminate the reaction. After the reaction mixture was centrifuged, the upper layer was collected, and DNAs were collected by ethanol precipitation and separated on 0.8% agarose gel. A DNA fragment of 8 kilobase pairs was collected by using EASY TRAP Ver. 2 (DNA collection kit, Takara Shuzo). On the other hand, the tac promoter region was amplified by PCR using the pKK223-3 plasmid (expression vector, Pharmacia) as a template and the primers shown in SEQ ID NOS: 17 and 18 (a cycle consisting of denaturation at 94° C. for 20 seconds, annealing at 55° C. for 30 seconds and extension reaction at 72° C. for 60 seconds was repeated for 30 cycles). Pyrobest DNA polymerase (Takara Shuzo) was used for PCR. The DNA fragment containing the amplified tac promoter was purified by using PCR prep (Promega) and then digested at the restriction enzyme sites preliminarily designed in the primers, i.e., at EcoRI and EcoT22I sites. Then, the reaction mixture was added to a phenol/chloroform solution and mixed to terminate the reaction. After the reaction mixture was centrifuged, the upper layer was collected, and DNAs were collected by ethanol precipitation and separated on 0.8% agarose gel. A DNA fragment of about 0.15 kbp was collected by using EASY TRAP Ver. 2.

The digestion product of the pRS vector prepared as described above and the tac promoter region fragment were ligated by using DNA Ligation Kit Ver. 2 (Takara Shuzo). This ligation reaction solution was used to transform Escherichia coli (E. coli JM109 competent cells, Takara Shuzo). The cells were plated on LB agar medium containing 20 mg/L of streptomycin and incubated overnight at 37° C. The colonies that appeared on the agar medium were each inoculated into LB liquid medium containing 20 mg/L of streptomycin and cultured at 37° C. for 8 hours with shaking. The plasmid DNA was extracted from each culture broth by the alkali-SDS method, and structure of each plasmid was confirmed by digestion with restriction enzymes to obtain pRStac. A plasmid in which the transcription directions of the streptomycin resistance gene on the pRS vector and the tac promoter were identical to each other was selected as pRStac.

By using the plasmid pRS-orf#3098 prepared as described above or pRStac as a control plasmid, the DLC10 strain was transformed by electroporation and selected on the SEII agar medium (containing 20 μg/ml of kanamycin, 50 μg/ml of streptomycin and 1 g/l of cadaverine).

When the selected DLC10/pRS-orf#3098 strain was inoculated into the SEII agar medium not containing cadaverine (containing 20 μg/ml of kanamycin and 50 μg/ml of streptomycin), growth of the pRStac-orf#3098 introduced strain was possible, whereas the DLC10/pRStac strain as the control strain could not grow. Furthermore, plasmids were extracted from the DLC10/pRS-orf#3098 strain by using Wizard Minipreps produced by Promega and confirmed by electrophoresis. As a result, it was confirmed that the strain harbored the intended plasmid, and therefore it was found that the protein encoded by orf#3098 on the plasmid acted in trans, and thereby the complementation was attained. It can be considered that the above results indicated that the deficiency of orf#3098 itself imparted the cadaverine auxotrophy for growth of the strain.

### Example 3

Complementation of orf#3098 Deficiency in Methylophilus methylotrophus by Introduction of ldcC Gene Derived from E. coli

(1) Preparation of Plasmid Carrying ldcC Gene Derived from E. coli

In order investigate whether an ldcC gene derived from E. coli could complement the cadaverine auxotrophy of the DLC10 strain for growth, a plasmid carrying ldcC derived from E. coli was prepared first. The E. coli W3110 strain was cultured overnight at 37° C. in the LB medium (10 g/l of trypton, 5 g/l of yeast extract, 10 g/l of NaCl), and a chromosomal DNA was prepared from the obtained cells by using Genomic DNA Purif. Kit produced by Edge BioSystems. This chromosomal DNA was used as a template together with DNA primers (PstI site was ligated to the 5′ end side) having the sequences shown as SEQ ID NOS: 15 and 16 (J. Bacteriol., 179 (14), 4486-4492 (1997)) to perform PCR (amplification reaction conditions: Pyrobest DNA polymerase produced by Takara Shuzo was used, a cycle consisting of denaturation at 98° C. for 10 seconds, annealing at 60° C. for 30 seconds and DNA strand extension reaction at 72° C. for 2 minutes was repeated for 25 cycles). The obtained DNA fragment having a size of about 2.3 kb was digested with the restriction enzyme PstI (Takara Shuzo). Separately, the vector pRStac was digested with Sse8387I, then subjected to a dephosphorylation treatment and ligated with the aforementioned PCR fragment (Ligation Kit Ver. 2 produced by Takara Shuzo was used). The plasmid carrying ldcC of E. coli prepared as described above was designated pRS-ldcC-F (carrying ldcC in the forward direction with respect to the tac promoter) or pRS-ldcC-R (carrying ldcC in the reverse direction with respect to the tac promoter).

(2) Confirmation of Complementation of orf#3098 Deficiency ofDLC1 Strain by LDC Derived from E. coli

The DLC10 strain was transformed with each of the both plasmids prepared as described above by electroporation, and transformants were selected on the SEII agar medium (containing 20 μg/ml of kanamycin, 50 μg/ml of streptomycin and 1 g/l of cadaverine). As a result, no transformant could be obtained with pRStac-ldcC-F, and a transformant could be obtained only with pRStac-ldcC-R.

This DLC10/pRStac-ldcC-R strain was applied to the SEII agar medium not containing cadaverine (containing 20 μg/ml of kanamycin and 50 μg/ml of streptomycin), and it was confirmed that the DLC10/pRStac-ldcC-R strain could grow, whereas the DLC10/pRS-tac strain as the control strain could not grow. This result indicates that LDC (lysine decarboxylase) of E. coli could complement the cadaverine auxotrophy of the orf#3098 deficient strain of Methylophilus methylotrophus.

### Example 4

Production of L-lysine by orf#3098 (ldc gene)-Disrupted Methylophilus methylotrophus Strain

(1) Construction of Plasmid pRSlysE24 for L-lysine Production

In order to introduce lysE gene which encodes a protein showing activity to excrete lysine in Corynebacterium glutamicum into a Methylophilus bacterium, a plasmid pRSlysE24 for expression of lysE was constructed by using pRStac mentioned above.

pRStac prepared in Example 2, (4) was digested with Sse83871 (Takara Shuzo) and SapI (New England Biolabs), and added to a phenol/chloroform solution and mixed to terminate the reaction. After the reaction mixture was centrifuged, the upper layer was collected, and DNAs were collected by ethanol precipitation and separated on 0.8% agarose gel to obtain a DNA fragment of about 9.0 kbp.

The lysE gene fragment was also amplified by PCR using a chromosome extracted from the Brevibacterium lactofermentum 2256 strain (ATCC 13869) as a template and the primers shown in SEQ ID NOS: 19 and 20 (denaturation at 94° C. for 20 seconds, annealing at 55° C. for 30 seconds and extension reaction at 72° C. for 90 seconds). Pyrobest DNA polymerase (Takara Shuzo) was used for PCR. The obtained fragment was purified by using PCR prep (Promega) and then digested with the restriction enzymes Sse83871 and SapI. The reaction mixture was added to a phenol/chloroform solution and mixed to terminate the reaction. After the reaction mixture was centrifuged, the upper layer was collected, and DNAs were collected by ethanol precipitation, purified on 0.8% agarose gel and collected.

The digestion product of the pRStac vector and the lysE gene region fragment prepared as described above were ligated using DNA Ligation Kit Ver. 2 (Takara Shuzo). This ligation reaction solution was used to transform Escherichia coli (E. coli JM109 competent cells, Takara Shuzo). The cells were plated on LB agar medium containing 20 mg/L of streptomycin and incubated overnight at 37° C. The colonies that appeared on the agar medium were each inoculated into LB liquid medium containing 20 mg/L of streptomycin and cultured at 37° C. for 8 hours with shaking. A plasmid DNA was extracted from each culture broth by the alkali-SDS method, and structure of the plasmid was confirmed by digestion with restriction enzymes and determination of nucleotide sequence to obtain pRSlysE. In pRSlysE, the lysE gene was positioned so that its transcription direction is the same as that of the tac promoter.

pRSlysE obtained as described above was introduced into Methylophilus methylotrophus AS1 strain (NCIMB 10515) by electroporation (Canadian Journal of Microbiology, 43, 197 (1997)). As a result, transformant could barely be obtained. Furthermore, when nucleotide sequences of plasmids extracted from several strains that could form colonies were examined, a mutation was introduced into the lysE gene. And when the colonies were cultured, L-lysine did not accumulate in the culture supernatants. However, when many colonies were further examined, a mutant-type lysE gene that could impart an ability to produce L-lysine to Methylophilus bacteria, i.e., that could function, could be obtained through analysis of pRSlysE introduced with a mutation.

This mutant lysE gene was designated as lysE24 gene. The nucleotide sequence of the lysE24 gene was analyzed, and it was found that the mutation did not result in amino acid substitution, but a nonsense mutation introducing a stop codon around the center of the translation region of lysE. The nucleotide sequence of the wild type lysE gene and the amino acid sequence encoded by it are shown as SEQ ID NOS: 21 and 22. In lysE24, T (thymine) was inserted after G (guanine) at position 355 of the wild-type lysE gene shown in SEQ ID NO: 21. The nucleotide sequence of lysE24 and the amino acid sequence encoded by it are shown as SEQ ID NOS: 23 and 24. This plasmid carrying lysE24 was designated pRSlysE24.

(2) Preparation of Plasmid pRSdapA having dapA * Gene

A plasmid was prepared having a gene encoding dihydrodipicolinate synthase that was not subject to feedback inhibition by L-lysine (dapA*) as an L-lysine biosynthesis system enzyme gene.

pRStac prepared in Example 2, (4) was digested with Sse83871 and XbaI, added to a phenol/chloroform solution and mixed with it to terminate the reaction. After the reaction mixture was centrifuged, the upper layer was collected, and DNAs were collected by ethanol precipitation and separated on 0.8% agarose gel to collect a DNA fragment of about 9 kbp.

The known plasmid RSFD80 (see WO90/16042) containing that gene was used as a template to amplify dapA* via PCR using the primers shown in SEQ ID NOS: 25 and 26 (denaturation at 94° C. for 20 seconds, annealing at 55° C. for 30 seconds and extension reaction at 72° C. for 60 seconds). Pyrobest DNA polymerase (Takara Shuzo) was used for PCR. The obtained dapA* fragment was purified by using PCR prep (Promega) and then digested with restriction enzymes Sse83871 and XbaI. The reaction mixture was added to a phenol/chloroform solution and mixed to terminate the reaction. After the reaction mixture was centrifuged, the upper layer was collected, and DNAs were collected by ethanol precipitation and separated on 0.8% agarose gel to collect a DAN fragment of about 0.1 kbp.

The digestion product of the pRStac vector and the dapA* gene region fragment prepared as described above were ligated by using DNA Ligation Kit Ver. 2 (Takara Shuzo). This ligation reaction solution was used to transform Escherichia coli (E. coli JM109 competent cells, Takara Shuzo). The cells were plated on LB agar medium containing 20 mg/L of streptomycin and incubated overnight at 37° C. The colonies that appeared on the agar medium were each inoculated into LB liquid medium containing 20 mg/L of streptomycin and cultured at 37° C. for 8 hours with shaking. Plasmid DNA was extracted from the culture broth by the alkali-SDS method and structure of the plasmid was confirmed by digestion with restriction enzymes and determination of nucleotide sequence to obtain a pRSdapA plasmid. In the pRSdapA plasmid, the dapA* gene was positioned so that its transcription direction is the same as that of the tac promoter.

(3) Construction of Plasmid pRSlysEdapA having lysE24 Gene and dapA* Gene

A plasmid consisting of the pRSlysE24 plasmid inserted with the dapA* gene was constructed to evaluate effect of combining lysE24 and dapA*.

pRSlysE24 prepared in Example 4, (1) was digested with a restriction enzyme SapI and blunt-ended by using DNA Blunting Kit (Takara Shuzo). Furthermore, the plasmid pRSdapA prepared in Example 4, (2) was digested with restriction enzymes EcoRI and SapI, and a fragment of about 1 kbp containing tac promoter and dapA* region was separated on 0.8% agarose gel. This fragment was collected by using EASY TRAP Ver. 2 (Takara Shuzo). This fragment was blunt-ended as described above and ligated to the aforementioned digestion product of pRSlysE24 by using DNA Ligation Kit Ver. 2 (Takara Shuzo).

The aforementioned ligation reaction solution was used to transform Escherichia coli (E. coli JM109 competent cells, Takara Shuzo). The cells were plated on LB agar medium containing 20 mg/L of streptomycin and incubated overnight at 37° C. The colonies that appeared on the agar medium were each inoculated into LB liquid medium containing 20 mg/L of streptomycin and cultured at 37° C. for 8 hours with shaking. Plasmid DNA was extracted from this culture broth by the alkali-SDS method, and the structure of the plasmid was confirmed by digestion with restriction enzymes and determination of nucleotide sequence to obtain a pRSlysEdapA plasmid. In this plasmid, the lysE24 gene and the dapA* gene were positioned so that their transcription direction is the same.

The E. coli JM109 strain transformed with the pRSlysEdapA plasmid was designated AJ13832, and this strain was deposited at the independent administrative agency, National Institute of Advanced Industrial Science and Technology, International Patent Organism Depositary on Jun. 4, 2001 and received an accession number of FERM P-18371. Then, the deposit was converted to an international deposit under the provisions of the Budapest Treaty on May 13, 2002, and received an accession number of FERM BP-8042.

(4) Introduction of L-lysine Production Plasmid into orf#3098 (ldc) Deficient Strain of Methylophilus methylotrophus and L-lysine Production

The influence of the ldc gene deficiency on the L-lysine production of Methylophilus methylotrophus was investigated. First, since the DLC10 strain prepared in Example 2 was prepared from a wild-type strain, L-lysine-producing ability was not modified. Therefore, in order to effectively verify the influence of ldc deficiency on the L-lysine production, an ldc-disrupted strain was produced from the Methylophilus methylotrophus AS1 strain introduced with pRSlysEdapA in the same manner as that of Example 2, (2). The obtained strain was designated a DLC12/pRSlysEdapA strain.

The AS1/pRSlysEdapA strain as a control strain and the DLC12/pRSlysEdapA strain were applied to the SEII agar medium containing 50 μg/ml of streptomycin and the SEII agar medium containing 50 μg/ml of streptomycin and 1 g/l of cadaverine, respectively, and cultured overnight at 37° C. Then, the cells on about 3 cm² (square centimeters) of each medium surface were scraped, inoculated into 20 ml of the SEII production medium containing 1 g/l of cadaverine (containing 50 μg/ml of streptomycin) and cultured at 37° C. for 67 hours with shaking. After completion of the culture, the cells were removed by centrifugation, and the L-lysine concentration in the culture supernatant was determined by using an amino acid analyzer (Nihon Bunko, high performance liquid chromatography). As a result, the AS1/pRSlysEdapA strain accumulated 1.26 g/L of L-lysine in the medium, and the DLC12/pRSlysEdapA strain accumulated 1.79 g/L of L-lysine in the medium. Thus, it could be confirmed that the deficiency of ldc could improve the production of L-lysine.

While the invention has been described in detail with reference to preferred embodiments thereof, it will be apparent to one skilled in the art that various changes can be made, and equivalents employed, without departing from the scope of the invention. Each of the aforementioned documents, including the foreign priority document, JP 200347185, is incorporated by reference herein in its entirety.

1. A protein selected from the group consisting of:
(A) a protein which has the amino acid sequence of SEQ ID NO: 4;
(B) a protein which has the amino acid sequence of SEQ ID NO: 4 including substitution, deletion, insertion or addition of one or several amino acid residues and has lysine decarboxylase activity.

2. A protein selected from the group consisting of:
(A) a protein which has the amino acid sequence of SEQ ID NO: 4;
(B) a protein which has the amino acid sequence of SEQ ID NO:4 including substitution, deletion, insertion or addition of one or several amino acid residues,
whereby said protein has lysine decarboxylase activity and is at least 90% homologous to SEQ ID NO: 4.

3. A DNA encoding a protein selected from the group consisting of:
(A) a protein which has the amino acid sequence of SEQ ID NO: 4;
(B) a protein which has the amino acid sequence of SEQ ID NO: 4 including substitution, deletion, insertion or addition of one or several amino acid residues and has lysine decarboxylase activity.

4. A DNA encoding a protein selected from the group consisting of:
(A) a protein which has the amino acid sequence of SEQ ID NO: 4;
(B) a protein which has the amino acid sequence of SEQ ID NO: 4 including substitution, deletion, insertion or addition of one or several amino acid,
whereby said protein has lysine decarboxylase activity and is at least 90% homologous to SEQ ID NO: 4.

5. The DNA of claim 3, selected from the group consisting of:
(a) a DNA which has the nucleotide sequence of the nucleotide numbers 684 to 2930 in SEQ ID NO: 3;
(b) a DNA which is hybridizable with a DNA having the nucleotide sequence of the nucleotide numbers 684 to 2930 in SEQ ID NO: 3 under stringent conditions and which encodes a protein having lysine decarboxylase activity.

6. The DNA of claim 3, which is derived from a chromosome of a Methylophilus bacterium.

7. A Metlophilus bacterium which produces L-lysine and is modified so that intracellular lysine decarboxylase activity is reduced or eliminated.

8. A Methylophilus bacterium which produces L-lysine, wherein a gene on a chromosome having a nucleotide sequence identical to the DNA of claim 3 is disrupted, or a gene on a chromosome having homology to the DNA of claim 3 to such a degree that homologous recombination with the DNA occurs is disrupted, thereby expression of said gene is suppressed and the intracellular lysine decarboxylase activity is reduced or eliminated.

9. A method for producing L-lysine, comprising the steps of culturing the Methylophilus bacterium of claim 7 in a medium containg methanol as a major carbon source resulting in accumulation of L-lysine in culture, and collecting the L-lysine from the culture.
